(19) United States
(12) Patent Application Publication
(10) Pub. No.: US 2021/0045901 A1
(43) Pub. Date: Feb. 18, 2021

(54) EXPANDABLE MEDICAL DEVICES

(71) Applicant: Transit Scientific, LLC, South Salt Lake, UT (US)

(72) Inventors: Shawn P. Fojtik, Park City, UT (US); Greg Method, San Francisco, CA (US); Jennifer Arnold, Salt Lake City, UT (US)

(21) Appl. No.: 16/994,533

(22) Filed: Aug. 14, 2020

Related U.S. Application Data
(60) Provisional application No. 62/886,931, filed on Aug. 14, 2019.

(51) Int. Cl. A61F 2/91

(52) U.S. Cl. CPC A61F 2/91; A61F 2/9526; A61F 2210/0014

(57) ABSTRACT

Expandable medical devices, including implantable medical devices that expand, such as stents and anchors, may be selectively expanded to for treatment purposes, including to hold the expandable medical devices and/or other devices with which the expandable medical devices are used in place within the body of a subject (e.g., within a vessel, a duct, another tubular organ, etc.). Such an expandable medical device may include struts that rotate outwardly as the expandable medical device expands. As the struts rotate outwardly, they may engage a surface against which the expandable medical device is expanded, holding the expanded expandable medical device in place. The expandable medical device may also plastically deform upon while expanded, enabling it to remain in an expanding state once an expanding force is no longer applied thereto.

### CROSS-REFERENCE TO RELATED APPLICATION

A claim for priority to the Aug. 14, 2019 filing date of U.S. Provisional Patent Application No. 62/886,931, titled EXPANDABLE MEDICAL DEVICES (“the '931 Provisional Application”) is hereby made pursuant to 35 U.S.C. § 119(e). The entire disclosure of the '931 Provisional Application is hereby incorporated herein.

### TECHNICAL FIELD

This disclosure relates generally to expandable medical devices, including implantable medical devices that expand, such as stents and anchors. More specifically, this disclosure relates to expandable medical devices that may be selectively expanded to for treatment purposes, including to hold the expandable medical devices and/or other devices with which the expandable medical devices are used in place within the body of a subject (e.g., within a vessel, a duct, another tubular organ, etc.). This disclosure also relates to methods for deploying expandable, implantable medical devices.

### RELATED ART

Existing stents may be implanted within the body of a subject to treat plaques and lesions and to hold the passageways through vessels, ducts, or other tubular organs open. Typically, a stent is applied to a balloon of a percutaneous transluminal angioplasty (PTA) catheter or a percutaneous transluminal coronary angioplasty (PTCA) catheter, and then introduced into the body. Once the stent has been advanced to a desired location within the subject's body, the balloon may be inflated, deforming the stent and forcing it against the inner surfaces of the vessel or duct. The balloon is then deflated and removed from the subject's body, with the stent remaining in place.

Stents sometimes migrate, which may decrease or negate their effectiveness and which may be dangerous to the individuals within whom are placed. Biliary stents are particularly prone to migration. Migration may occur if a stent is too small for the location in which it is placed, because of movement of or within the hollow organ within which it is placed, or for other reasons. Additional procedures are typically required to remove and replace stents that have migrated, presenting further risks to the health of patients.

Stents that are too large may damage the organs within which they are placed.

Some stents have configurations that enable them to push against, or apply force to, the surfaces against which they are positioned. While existing stents may push against those surfaces, they lack features that enable them to safely score and/or securely engage the surfaces (e.g., by breaking the interelastic layer of a blood vessel (e.g., an artery, a vein, etc.).

### SUMMARY

An expandable implantable medical device according to this disclosure, which may also be referred to as an “expandable device” for the sake of simplicity, may have a configuration that prevents migration and, thus, enables it to remain in place within a subject's body. In various embodiments, such an expandable implantable medical device may comprise a stent, an anchor, or another device that can be held in place within a vessel, a duct, or another tubular organ. The expandable device may be tubular in shape, with a plurality of struts positioned around its outer surface. Each strut may extend along a length of the expandable device. The expandable device or a portion thereof may expands under an expanding force.

The expanding force may comprise a radially outward force from within the expandable device, which may apply tension to the expandable device in a radially outward direction; such tension may be referred to herein as “radial tension.” The expanding force may be applied by way of an expander (e.g., a balloon, etc.). Application of the expanding force may expand the expandable element, transitioning the expandable element from an unexpanded state to an expanded state.

Alternatively, an expandable device may self-expand. Self-expanding embodiments of expandable devices may be made with hypotubes formed from shape memory alloys. Such a hypotube may be expanded, laser cut, heat set, and collapsed (e.g., onto a mandrel, wire, etc., of desired dimension; etc.). A sheath or another cover may be applied to an exterior surface of the expandable device. In use, the expandable device may be introduced into a subject's body and, once it has been advanced to a desired location, the sheath or other cover may be removed. The subject's body temperature may then heat the material of the expandable element, causing it to expand.

As the expandable device or a portion thereof radially expands, portions of adjacent struts may be forced apart from one another and each strut may rotate (e.g., by up to about 90°, etc.), causing an edge or a corner of the expandable device to be somewhat radially disposed. When in a radially disposed orientation, the edge or corner of each strut may contact, engage, or even score a surface against which the strut of the expandable device is forced. By rotating to contact, engage, and/or score surfaces of the organ within which the expandable device is placed, the struts may secure the expandable device in place within the organ.

Struts may be defined by adjacent series of slits in the tube, with the slits of each series being offset from, or staggered relative to, the slits of a circumferentially adjacent series. Each series of slits may be positioned along a generator of the expandable device (i.e., a line extending from one end of the expandable device to the other end of the expandable device, parallel to an axis of the expandable device). Each slit may overlap, or be staggered relative to, about half of one slit (if the slit is located at or near an end of the expandable device) or two slits (if the slit is located intermediately along a length of the expandable device) of an adjacent series of slits; stated another way, the slits of an expandable device may have a so-called “brickwork” arrangement, or they may be arranged like the bricks in a so-called “running bond pattern.”

The slits may comprise linear slits through the wall of the tube. Shaped slits are also within the scope of this disclosure. Suitable shapes include thin diamond shapes, with each slit defining an opening with the shape of a single diamond, a pair of adjacent diamonds, etc. Such an arrangement may enable a portion of a strut to rotate (e.g., by about 45°, by about 90°, etc.) or cause rotation of that portion of the strut upon placement of that strut under radial tension; for example, by expanding a portion of the expandable device on which that strut is located. The portion of the expandable device may be expanded, for example, by expanding an expander that has been placed within the expandable device (e.g., by inflating a balloon within the expandable device, etc.). Such an arrangement, along with the material or materials from which the expandable device is formed, may enable the expandable device to plastically deform and, thus, maintain its expanded arrangement once any radial tension (e.g., from an internal force, etc.) on that portion of the expandable device is released. The radial tension may be released, for example, by contracting an expander within the expandable device (e.g., by deflating a balloon within the expandable device, etc.).

As the struts rotate, they may engage the surfaces against which they are positioned, which may hold the expanded device in place. Additionally, a configuration of the expandable device may make it dynamically flexible, which may enable it to remain in place as the organ within which it is positioned moves or changes.

The expandable device may carry a medicament, which may be delivered to the surface that is engaged or scored while that surface is engaged or scored. The medicament may be carried by inner surfaces of the expandable device (e.g., coated onto the inner surfaces of the struts, etc.), thereby protecting the medicament and preventing its introduction into the body of a subject until the stent has been advanced to the location where the medicament is to be administered. Such a configuration may be useful for delivering drugs that may be harmful or present mortality issues when improperly administered (e.g., paclitaxel, etc.). Upon expansion of the expandable device or a portion thereof and causing one or more struts of the expandable device to score that surface, the medicament may be delivered to the surface and into the score marks that have been formed.

A method according to this disclosure includes advancing an expandable device to a desired location within a hollow organ of a subject's body and expanding the expandable device to an extent sufficient to cause struts thereof to rotate outwardly and engage (e.g., contact, become embedded in, score, etc.) interior surfaces of the hollow organ, anchoring the expandable device and, optionally, another device (e.g., an existing stent, etc.) in place within the hollow organ. Rotation of the struts may also increase the column strength of the expandable device. Expansion of the expandable device may also be sufficient to plastically deform the expandable device, which may prevent the expandable device from contracting once any an expanding force is removed therefrom. The expandable device may expanded further until its size is appropriate for the location where the expandable device is placed).

The extent to which the struts of the expandable device rotate upon expansion of the expandable device depends in part upon the material from which the expandable device is formed and in part upon the extent to which the slits of the expandable device are opened upon expanding the expandable device. As an example, a strut may start to rotate when expansion opens, or increases, angles at the ends of the slits that define the strut by at least about 5° to at least about 15°.

The extent to which the shape of the expandable device is plastically deformed following expansion and release of an expanding force also depends in part upon the material from which the expandable device is formed and in part upon the extent to which the slits are opened upon expanding the expandable device. As an example, plastic deformation of the expandable device may not occur until expansion opens, or increases, angles at the ends of the slits by at least about 25° to at least about 40°.

If the expandable device is not expanded to or beyond its nonplastic deformation limit, releasing the expanding force may enable the expandable device to contract, in some embodiments to substantially its original shape and dimensions. Thus, the expandable device may be repositioned within or removed from a subject's body prior to being expanded beyond its nonplastic deformation limit.

If the expandable device is expanded to or beyond its nonplastic deformation limit, the expandable device may remain expanded when the expanding force and the expander are removed from the expandable device.

Other aspects of this disclosure, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 provides a partial view of an embodiment of an expandable device according to this disclosure;

FIG. 2 shows an embodiment of an expandable device in an expanded state, or arrangement;

FIG. 3 shows the embodiment of expandable device of FIG. 2 remaining in the expanded state upon removal of a radially outward force and, thus, a radial tension, therefrom;

FIG. 4 is a cross-sectional view showing an embodiment of an expandable device according to this disclosure in an unexpanded state, within an organ, such as a blood vessel or a duct;

FIG. 5 is a cross-sectional view showing the embodiment of expandable device depicted by FIG. 4 in an expanded state within the organ;

FIG. 6 includes a table of data corresponding to five (5) different embodiments of expandable devices according to this disclosure expanded to nine (9) different diameters, with the data including increases in the angles at the ends of the slits of the expandable devices; FIG. 6 also includes a chart plotting the increases in angles (y-axis) against the diameter of an expander (a PTA balloon) used to expand the expandable devices;

FIG. 7 schematically depicts another embodiment of expandable device according to this disclosure, the expandable device including at least one spine that limits movement of the expandable device; and

FIG. 8 schematically depicts a variation of the embodiment of expandable device illustrated by FIG. 7, in which a first set of spines limits movement of a first portion the expandable device in a first plane and a second set of spines limits movement of a second portion of the expandable device in a second plane.

### DETAILED DESCRIPTION

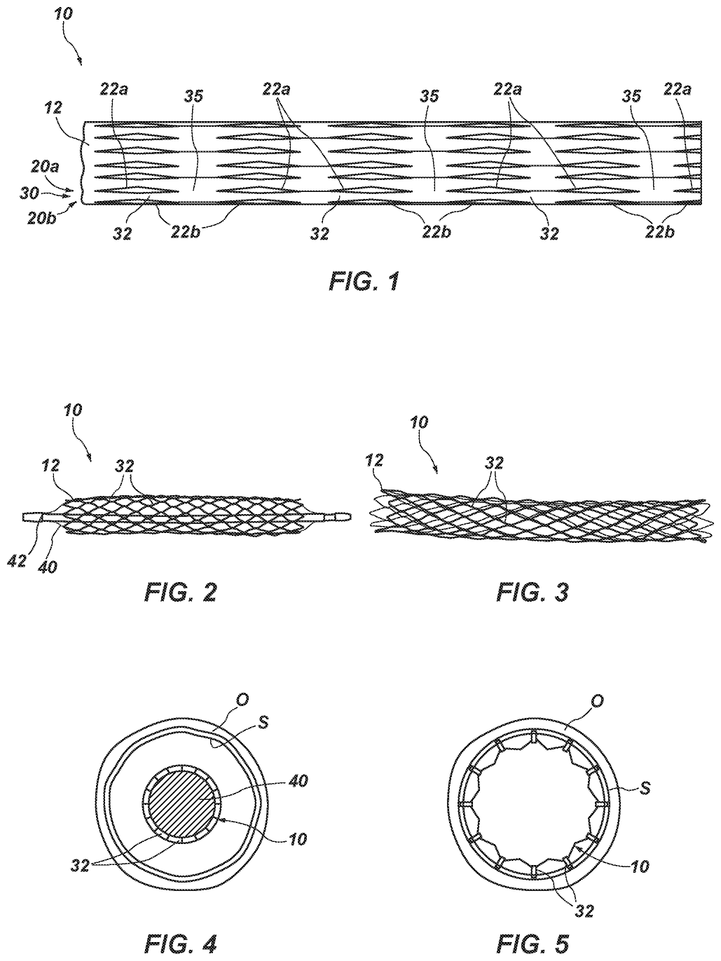

FIG. 1 illustrates an embodiment of an expandable device 10 that includes a tubular body 12 with series 20 of slits 22 defined therein (e.g., by laser cutting techniques, etc.) and series 30 of struts 32 defined between circumferentially adjacent series 20 of slits 22. As illustrated, each series 20 of slits 22 is oriented longitudinally, along a generator the tubular body 12 of the expandable device 10 (i.e., a line extending from one end of the tubular body 12 to the other end of the tubular body 12, parallel to an longitudinal axis through the center of the tubular body 12. Alternatively, each series 20 of slits 22 may be somewhat helically oriented around the tubular body 20.

The tubular body 12 of the expandable device 10 may have any of a variety of dimensions. Without limitation, the tubular body 12 may be as small as about 3 French (F) (a 1 mm outer diameter (OD)) or less. Tube with sizes of 4 F (1.333 mm outer diameters) and less may also be used to manufacture an expandable device 10 according to this disclosure, as may larger tubes. A thickness of the tubular body 12 and, thus, of each strut 32 defined from the tubular body 12 may be about 0.001 inch (about 0.0254 mm), about 0.002 inch (about 0.0508 mm), or larger.

In a specific embodiment, the tubular body 12 of the expandable device 10 may comprise or be defined from a tube (e.g., a hypotube, etc.) formed from a substantially rigid material, such as a metal (e.g., stainless steel (e.g., 316L stainless steel, 316 stainless steel, etc.), a memory metal (e.g., nitinol, etc.), cobalt chromium (CoCr), a nickel chromium (NiCr or nichrome) alloy (including, without limitation, NiCr steel), etc.). Alternatively, the tubular body 12 may comprise a polymer. A suitable polymer may have a sufficient hardness (e.g., at least 35 Shore D, 35 Shore D to 55 Shore D, 35 Shore D to 72 Shore D, etc.). Examples of suitable polymers include, but are not limited to polyether ether ketone (PEEK), polyimide, nylon, polyether block amides (PEBA, such as that branded as PEBAX®), and extruded plastics, provided that they have a wall thickness that does not exceed the width of their struts 32.

The slits 22 may comprise linear slits through the wall of the tube. Shaped slits 22 are also within the scope of this disclosure. Suitable shapes include thin diamond shapes, with each slit 22 defining an opening with the shape of a single diamond, a pair of adjacent diamonds, etc. FIG. 1 shows an embodiment of an expandable device 10 with double diamond shaped slits 22.

Each slit 22a may overlap, or be staggered relative to, about half of one slit 22b (if the slit 22a is located at or near an end of the tubular body 12 of the expandable device 10) or two slits 22b (if the slit 22a is located intermediately along a length of the tubular body 12 of the expandable device 10) of an adjacent series 20b of slits 22b. Stated another way, the slits 22 of an expandable device 10 may have a so-called “brickwork” arrangement, or they may be arranged like the bricks in a so-called “running bond pattern.” Alternatively, circumferentially adjacent slits 22a and 22b may be aligned or substantially aligned with one another.

Solid regions between longitudinally adjacent slits 22 may define a spine 35 that extends longitudinally or helically over the circumference of the tubular body 12 of the expandable device 10.

Each strut 32 of an expandable device 10 with an arrangement of the type depicted by FIG. 1 may be able to rotate (e.g., by about 45°, by about 90°, etc.). For example, expanding a portion of the expandable device 10 on which a strut 32 is located may cause the strut 32 to at least partially rotate. FIG. 2 illustrates expansion of a portion of the expandable device 10 by placing an expander 40 within an interior of the tubular body 12 of the expandable device 10. Without limitation, the expander 40 may comprise a balloon of a PTA catheter or a PTCA catheter 42. As the expander 40 expands (e.g., by inflating a balloon within the expandable device 10, etc.), radial tension is introduced into the portion of the expandable device 10 within which the expander 40 resides.

In some embodiments, such as that depicted by FIG. 3, an expandable element 10 with such an arrangement, along with the material or materials from which the expandable device 10 is formed, may enable the expandable device 10 to plastically deform and, thus, maintain its expanded arrangement once any radial tension (e.g., from an internal force applied by an expander 40 (FIG. 2), etc.) on that portion of the expandable device 10 is released. The radial tension may be released, for example, by contracting the expander 40 (FIG. 2) within the expandable device 10 (e.g., by deflating a balloon within the tubular body 12 of the expandable device 10, etc.).

As illustrated by FIG. 4, the expandable device 10 may be introduced to a desired site within an organ O while the expandable device 10 is in its unexpanded state. In the unexpanded state, the struts 32 of the expandable device 10 are circumferentially oriented with respect to a remainder of the expandable device 10 and, thus, the struts 32 have not rotated yet. With the expandable device 10 at the desired site within the organ O an expander 40 within the expandable device 40 may expand the expandable device 10 from its unexpanded state to an expanded state, such as that shown in FIG. 5. As the expandable device 10 expands, its struts 32 rotate. As the struts 32 rotate, they may engage the surfaces S against which they are positioned, which may hold the expanded expandable device 10 in place within the organ O. Additionally, a configuration of the expandable device 10 may make it dynamically flexible, which may enable it to remain in place as the organ O within which it is positioned moves or changes.

In embodiments where the expandable device 10 is formed from a 6F 316L stainless steel hypotube, the struts 32 will rotate when the expandable device 10 is expanded enough to cause the ends of the slits 22 to open to an angle of at least about 15°. As long as the ends of the slits 22 open to a nonplastic deformation limit of an angle of about 48° or more, plastic deformation occurs. Thus, the expandable device 10 may remain in its expanded state with struts 32 rotated upon releasing an expanding force from the expandable device 10, as depicted by FIG. 5.

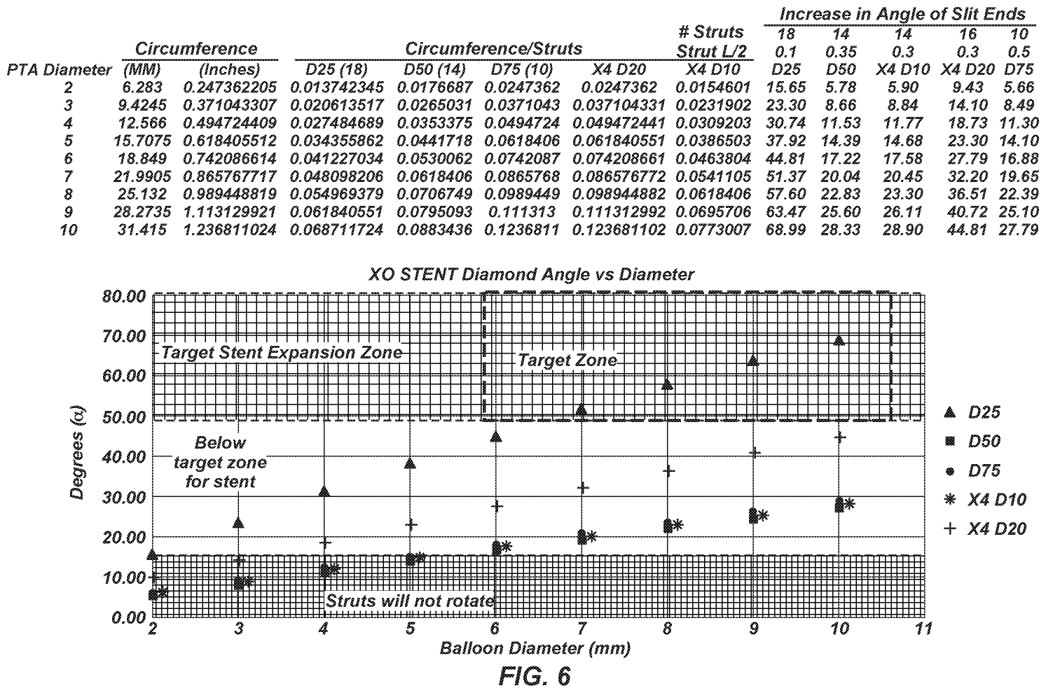

Turning now to FIG. 6, a table provides data for five (5) configurations of 6F (2 mm OD) 316L stainless steel expandable devices with double diamond slits (as shown in FIG. 1) and identify the extent to which expansion of each expandable device by PTA balloon catheters of a plurality of different sizes, or outer diameters, increases the angles at the ends of the slits of the expandable device. The calculations provided in the last five (5) columns of the table identify the angles that an expandable device with a certain number of struts and struts of a set length will open to when the expandable device is expanded to various specific inner diameters (e.g., with PTA balloon catheters, etc.).

The chart of FIG. 6 identifies the amount of expansion needed to cause the struts of each embodiment of expandable device to rotate, as well as the nonplastic deformation limit of each embodiment of expandable device. Based on these factors, a determination can be made of whether expansion of an expandable device of a particular design will, based on the material from which the expandable device is defined (e.g., the material from which the hypotube is formed, etc.), cause the struts of the expandable device to rotate and whether the expandable device will be non-plastically deformed (i.e., able to return substantially to its unexpanded state) or plastically deformed (i.e., unable to return substantially to its unexpanded state).

The angle (a) calculations for each unit of expansion (e.g., 1 mm, 2 mm, 3 mm, 4, mm, 5 mm, 6 mm, 7 mm, etc.), as set forth in the first column of the table of FIG. 6, are made by taking the arc tangent of the quotient of the width (w), or arc length, of each strut divided by half the length (l) of the strut (½), and then multiplying that value by the product of 180/π and 2 (or 360/π), as follows:

α=atan(w/(½)*180/π*2.

Calculations have been made and are illustrated for five different expandable devices defined from 6F (2 mm OD) 316L stainless steel hypotubes. A first of the expandable devices D25 includes 18 struts around each location of the circumference of the expandable device D25. Each strut, which has a width of about 0.014 inch (about 0.356 mm) and is capable of being embedded into a surface to a depth of up to 0.007 inch (about 0.178 mm), is defined by a pair of diamond shaped slits and has a length of 0.2 inch (about 5 mm). Longitudinally adjacent slits are spaced about 0.014 inch (about 0.356 mm) apart from each other.

A second of the expandable devices D50 includes 14 struts around each location of the circumference of the expandable device D50. Each strut, which has a width of about 0.018 inch (about 0.450 mm) and is capable of being embedded into a surface a depth of about 0.009 inch (about 0.225 mm), is defined by a pair of diamond shaped slits and has a length of 0.7 inch (about 17.8 mm). Longitudinally adjacent slits 32 are spaced about 0.018 inch (about 0.450 mm) apart from each other.

A third of the expandable devices X4 D10 includes 14 struts around each location of the circumference of the expandable device X4 D10. Each strut is defined by a pair of diamond shaped slits and has a length of 0.6 inch (about 15.3 mm). Longitudinally adjacent slits 32 are spaced about 0.0155 inch (about 0.391 mm) apart from each other.

A fourth of the expandable devices X4 D20 includes 16 struts around each location of the circumference of the expandable device X4 D20. Each strut is defined by a pair of diamond shaped slits and has a length of 0.6 inch (about 15.3 mm). Longitudinally adjacent slits 32 are spaced about 0.0247 inch (about 0.627 mm) apart from each other.

A fifth of the expandable devices D75 includes 10 struts around each location of the circumference of the expandable device D75. Each strut, which has a width of about 0.0247 inch (about 0.627 mm) and is capable of being embedded up to 0.0124 inch (about 0.315 mm), is defined by a pair of diamond shaped slits and has a length of 1.0 inch (about 25.4 mm). Longitudinally adjacent slits 32 are spaced about 0.0247 inch (about 0.627 mm) apart from each other.

As the graph of FIG. 6 depicts, the expandable device D25 must be expanded to an inner diameter of about 2 mm (e.g., with a PTA balloon catheter, etc.) or more to cause its struts to rotate, while expandable devices X4 D20 must be expanded to an inner diameter of about 3.5 mm (e.g., with a PTA balloon catheter, etc.) or more to cause its struts to rotate, and the expandable devices D50, D75, and X4 D10 must be expanded to inner diameters of about 6 mm (e.g., with a PTA balloon catheter, etc.) or more to cause their struts to rotate.

When the expandable device is expanded beyond its plastic deformation limit, it may remain expanded following the removal of radial tension therefrom (e.g., deflation of a balloon, etc.). The non-plastic deformation limit of the expandable device D25 may be reached upon expanding an inner diameter of the expandable device D25 to about 7 mm or more. By extrapolating the data in FIG. 6, the non-plastic deformation limit of the expandable device X4 D20 may be reached upon expanding an inner diameter off the expandable device X4 D20 to about 11 mm or more. An expandable device according to this disclosure may include relatively short struts (e.g., struts that are about 0.5 inch (about 12.7 mm) long or shorter, about 0.4 inch (about 10.2 mm) long, about 0.3 inch (about 7.62 mm) long, 0.25 inch (about 6.35 mm) long, about 0.2 inch (about 5.1 mm) long, about 0.15 inch (about 3.81 mm) long, about 0.1 inch (about 2.54 mm) long, etc.) and slits (e.g., slits that are less than about 0.5 inch (about 12.7 mm) long, less than about 0.4 inch (about 10.2 mm) long, less than about 0.3 inch (about 7.62 mm) long, less than about 0.25 inch (about 6.35 mm) long, less than about 0.2 inch (about 5.1 mm) long, less than about 0.15 inch (about 3.81 mm) long, less than about 0.1 inch (about 2.54 mm) long, etc.).

Expandable devices X4 D20, D50, D75, and X4 D10 may be useful applications where resilient contraction of the expandable device is desirable (e.g., as the expandable section of an exoskeleton device, such as those disclosed by U.S. patent application Ser. No. 16/540,046, filed on Aug. 13, 2019 and titled EXOSKELETON DEVICE WITH EXPANDABLE SECTION FOR SCORING, the entire disclosure of which is hereby incorporated herein, etc.).

When fully expanded, the outer diameter of the expandable device may increase by about 3 times to 4 times or more. In some embodiments, the outwardly rotated struts, which may be embedded within the interior surfaces of the hollow organ within which the expandable device has been placed, may securely anchor the expandable device in place within the hollow organ. These may include the struts of the expandable devices D25 and X4 D20.

In embodiments where an expanding force is applied to interior surfaces of the expandable device, an expander (e.g., a balloon, etc.) that applies the expanding force may be contracted (e.g., deflated, etc.) and then removed from the expandable device, from the hollow organ, and from the subject's body.

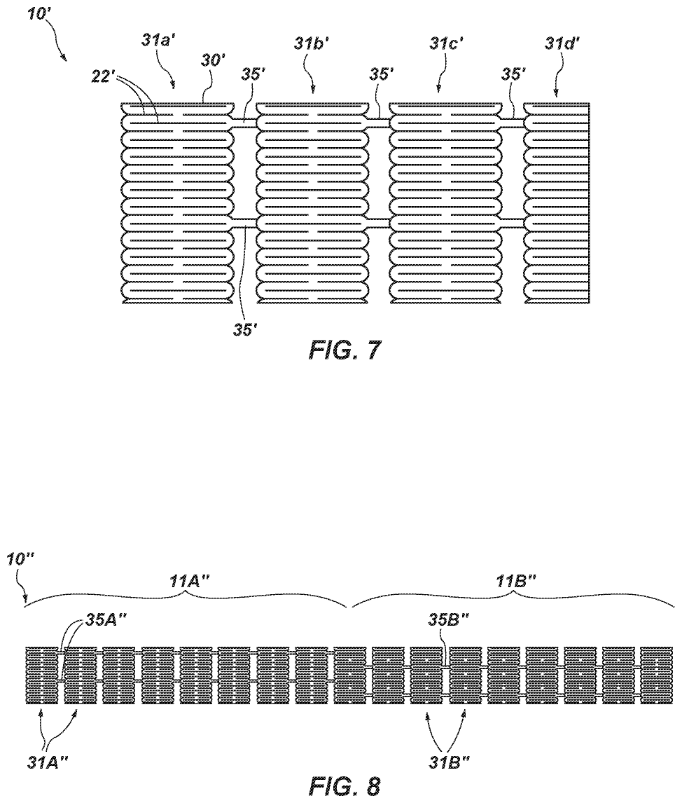

Turning now to FIG. 7, another embodiment of expandable device 10′ is depicted. The slits 22′ of circumferentially adjacent series 20′ of the expandable device 10′ are staggered, but define circumferentially oriented columns 31′ of struts 30′. Longitudinally adjacent columns 31a′ and 31b′ of struts 32′ may be secured to one another with spines 35′. The spines 35′ may extend longitudinally or helically over the circumference of the expandable device 10′. The spines 35′ may be positioned to enable limit flexion of the expandable device 10; for example, to only allow the expandable device 10′ to be flexed in specific directions, or degrees of freedom. As an example, an expandable device 10′ may include spines 35′ that enable it to readily flex, or bend, two opposite directions within a single plane, or within two degrees of freedom. Alternatively, the spines 35′ may allow the expandable device 10′ to flex in three directions, in four directions, etc.

As illustrated by FIG. 7, two spines 35′ are located between adjacent columns 31′. These spines 35′ may provide two degrees of freedom, meaning that, when expanded, or deployed, the expandable device 10′ may only be able to flex in one plane. The numbers and locations of the spines 35′ may be designed to longitudinally strengthen or support the expandable device 10′, limiting its ability to flex to predetermined directions.

FIG. 8 depicts an embodiment of expandable device 10″ that includes first pairs of spines 35A″ in a first arrangement between adjacent first sets of columns 31A″ and second pairs of spines 35B″ in a second arrangement between adjacent second sets of columns 31B″. The first pairs of spines 35A″ in the first arrangement may allow a corresponding first portion 11A″ of the expandable device 10″ to flex in a first plane, while the second pairs of spines 35B″ in the second arrangement may allow a corresponding second portion 11B″ of the expandable device 10″ to flex in a second plane that intersects the first plane (e.g., perpendicularly, non-perpendicularly, etc.). Thus the first pairs of spines 35A″ may provide the first portion 11A″ with two degrees of freedom that differ from the two degrees of freedom imparted to the second portion 11B″ by the second pairs of spines 35B″. As a non-limiting example, the first pairs of spines 35A″ could enable the first portion 11A″ to move in the plane of FIG. 8 (i.e., up and down), while the second pairs of spines 35B″ could enable the second portion 11B″ to move in a plane oriented horizontally and perpendicular to FIG. 8 (i.e., into FIG. 8 and out of FIG. 8).

Various embodiments of expandable devices according to this disclosure may carry a medicament, which may be delivered to the surface that is engaged or scored while that surface is engaged or scored. The medicament may be carried by inner surfaces of the expandable device (e.g., coated onto the inner surfaces of the struts, etc.) or onto the edges of the struts of the expandable device, thereby protecting the medicament and preventing its introduction into the body of a subject until the stent has been advanced to the location where the medicament is to be administered. Such a configuration may be useful for delivering drugs that may be harmful or present mortality issues when improperly administered (e.g., paclitaxel, etc.). Upon expansion of the expandable device or a portion thereof and causing one or more struts of the expandable device to score that surface, the medicament may be delivered to the surface and into the score marks that have been formed.

With returned reference to FIGS. 4 and 5, a method according to this disclosure includes advancing an expandable device 10 to a desired location within a hollow organ O of a subject's body. Once the expandable device 10 reaches the desired location within the hollow organ O, it may be expanded. Expansion of the expandable device 10 may be effected with an expander 40 (e.g., a balloon of a PTA catheter or a PTCA catheter 42 (FIG. 2)). The expandable device 10 may be expanded to an extent sufficient to cause struts 32 thereof to rotate outwardly and contact, engage, and/or score interior surfaces S of the hollow organ O or surfaces of substances lining the interior surfaces of the hollow organ O. As the struts 32 contact, engage, and/or score surfaces, they may anchor the expandable device 10 and, optionally, another device (e.g., an existing stent, etc.) in place within the hollow organ O. Rotation of the struts 32 may also increase the column strength of the expandable device 10. Expansion of the expandable device 10 may also be sufficient to plastically deform the expandable device 10, which may prevent the expandable device from contracting once any expanding force is removed therefrom. The expandable device 10 may expanded further until its size is appropriate for the location where the expandable device 10 is placed).

As indicated previously herein, an expandable device 10 according to this disclosure may comprise a stent. Alternatively, an expandable device 10 according to this disclosure may be integrated into or secured to another implantable medical device to anchor that implantable medical device (e.g., a temporary implantable medical device, a permanently implantable medical device, etc.) to a particular location within a subject's body. One or more expandable devices 10 with struts 32 may be attached to the implantable medical device and deployed (e.g., with an internal force, such as that applied by an expander 40, such as a balloon; by self-expansion, such as by manufacturing the expandable device from a shape memory alloy; etc.) to prevent migration of the implantable medical device. Without limitation, an expandable device may be secured to one end or to each end of the implantable medical device (e.g., opposite ends of a linear or curvilinear device, all ends of a branched device, etc.). Implantable medical devices that include expandable devices 10 according to this disclosure are also within the scope of this disclosure.

In other embodiments, an expandable device 10 according to this disclosure may be used as a stent and/or as a stent scaffold for other devices, such as percutaneous valves, inferior vena cava filters, heart pumps (e.g., the IMPELLA® heart pump available from AbioMed, etc.), and the like.

An expandable device 10 according to this disclosure may also be used as the expandable section of an exoskeleton device, such as those disclosed by U.S. patent application Ser. No. 16/540,046, filed on Aug. 13, 2019 and titled EXOSKELETON DEVICE WITH EXPANDABLE SECTION FOR SCORING (U.S. Patent Application Publication US 2020/0069922 A1), the entire disclosure of which is hereby incorporated herein. The expandable section of such a device may be located at a distal end of the device (e.g., with a length of about 2 cm, 3 cm, 4 cm, 5 cm, 6 cm, etc.), with more proximal portions of the device comprising a catheter. The catheter may comprise a hypotube catheter, such those disclosed by U.S. patent application Ser. No. 16/748,541, filed on Jan. 21, 2020 and titled HYPOTUBE CATHETERS (U.S. Patent Application Publication US 2020/0230359 A1), the entire disclosure of which is hereby incorporated herein. In a variation of such a device, a proximal portion of the catheter (e.g., about 80-90% of the proximal-most portion of the length of the device, etc.) may be cut away, leaving just a wire (e.g., a burnished and polished flat wire, etc.).

Although the foregoing description provides many specifics, these should not be construed as limiting the scopes of any of the appended claims, but merely as providing information pertinent to some specific embodiments that may fall within the scopes of the appended claims. Features from different embodiments may be employed in combination. In addition, the scopes of the appended claims may encompass other embodiments. All additions to, deletions from, and modifications of the disclosed subject matter that fall within the scopes of the claims are to be embraced by the claims.

What is claimed:

1. An expandable device, comprising:
a tubular body including a plurality of series of slits, each series of slits including a plurality of slits extending substantially longitudinally along the expandable section, adjacent slits of the plurality of slits spaced apart slits of circumferentially adjacent series of slits of the plurality of series of slits defining a series of struts between the circumferentially adjacent series of slits, the tubular body, slits, and struts of the expandable device imparting the expandable device with an ability expand under an expanding force, causing the struts to rotate outwardly and the expandable device to plastically deform in such a way that the expandable device maintains an expanded state upon removal of the expanding force.

2. The expandable device of claim 1, wherein an outer surface of the expandable device is substantially smooth when the expandable device is in an unexpanded state.

3. The expandable device of claim 1, wherein the series of slits are arranged around a circumference of the expandable section along a plurality of generators of the circumference of the expandable section.

4. The expandable device of claim 1, wherein each series of slits of is longitudinally offset relative to an adjacent series of slits of the plurality of series of slits.

5. The expandable device of claim 1, wherein at least some slits of the plurality of slits of the series of slits of the plurality of series of slits have smooth diamond shapes defining an opening through the expandable section.

6. The expandable device of claim 5, wherein at least some slits of the plurality of slits of the series of slits of the plurality of series of slits have double diamond shapes.

7. The expandable device of claim 5, wherein each slit of the plurality of slits of the series of slits of the plurality of series of slits has a smoot diamond shape defining an opening through the expandable section.

8. The expandable device of claim 5, wherein a strut begins to rotate upon increasing angles that define ends of slits that define the strut by at least about 15°.

9. The expandable device of claim 5, wherein a strut begins to rotate upon increasing angles that define ends of slits that define the strut by at least about 5°.

10. The expandable device of claim 5, wherein the expandable device will plastically deform when the angles that define the ends of the slits that define the strut are increased by at least about 25°.

11. The expandable device of claim 5, wherein the expandable device will plastically deform when the angles that define the ends of the slits that define the strut are increased by at least about 40°.

12. The expandable device of claim 1, carrying a medicament on an interior surface or an edge of at least one strut.

13. A medical system, comprising:
an expandable device including a tubular body including a plurality of series of slits, each series of slits including a plurality of slits extending substantially longitudinally along the expandable section, adjacent slits of the plurality of slits spaced apart slits of circumferentially adjacent series of slits of the plurality of series of slits defining a series of struts between the circumferentially adjacent series of slits, the tubular body, slits, and struts of the expandable device imparting the expandable device with an ability expand under an expanding force, causing the struts to rotate outwardly and the expandable device to plastically deform in such a way that the expandable device maintains an expanded state upon removal of the expanding force; and
a medical device to be held in place by the expandable device.

14. The medical system of claim 13, wherein the medical device comprises an implantable medical device.

15. A method for securing a medical device in place within a body of a subject, comprising:
introducing an expandable device into a body of a subject to a location adjacent to which the medical device is to be secured;
expanding the expandable device, with slits of the expandable device opening such that angles at ends of each slit of the slits increase by at least about 5° to cause struts defined by the slits to rotate, forcing the struts of the expandable section of the exoskeleton device against the surface that is to be scored, and scoring the surface, and then increase by at least about 25° to cause the expandable device to plastically deform and to remain in place at the location adjacent to which the medical device is to be secured.

16. The method of claim 15, further comprising:
removing an expanding force from the expandable device with the expandable device remaining in an expanded state.

17. The method of claim 15, wherein the expandable device comprises the medical device to be secured in place adjacent to the location within the body of the subject.

18. The method of claim 15, further comprising:
securing the medical device to the expandable device.

19. The method of claim 15, wherein expanding the expandable device comprises increasing the angles at the ends of each slit by at least about 15° to cause the struts to rotate.

20. The method of claim 15, wherein expanding the expandable device comprises increasing the angles at the ends of each slit by at least about 40° to cause the expandable device to plastically deform.
